(19) United States
(12) Patent Application Publication
(10) Pub. No.: US 2018/0052130 A1
(43) Pub. Date: Feb. 22, 2018

(54) SENSOR DEVICE AND METHODS

(71) Applicant: NANO TERRA INC., Cambridge, MA (US)

(72) Inventors: Hootan Farhat, Somerville, MA (US); Kateri Paul, Medford, MA (US); Mitch Zakin, Andover, MA (US); Shekar Shetty, Newton, MA (US); Phil Graf, Chestnut Hill, MA (US); Piercen Oliver, Malden, MA (US); Joe McLellan, Quincy, MA (US)

(21) Appl. No.: 15/422,375

(22) Filed: Feb. 1, 2017

Related U.S. Application Data
(60) Provisional application No. 62/289,710, filed on Feb. 1, 2016.

(51) Int. Cl. G01N 27/12; G01N 33/00; G01N 27/07

(52) U.S. Cl. CPC G01N 27/126; G01N 33/5438; G01N 27/07; G01N 33/0057

(57) ABSTRACT

A device and method for detecting presence of a hazardous material in an environment or through a testing material, such as for example, a protective material. The device may include a sensor for detecting presence of the hazardous material. The sensor may comprise or be formed from a conductive polymer, a semi-conductive polymer or an electroactive polymer, the sensor being chemically reactive with the hazardous material to generate a change in electrical resistance in the sensor. The device may further include one or more conductive electrodes attached to the sensor configured to detect a change in resistance in the sensor, and a resistance measuring device electronically connected to the one or more electrodes for receiving data from the one or more electrodes and generating an output based on the data corresponding to an amount of hazardous material detected by the sensor in real-time.

### PRIORITY CLAIM

This application claims priority to U.S. Provisional Patent Application Ser. No. 62/289,710 filed Feb. 1, 2016, the entire contents of which is hereby incorporated by reference herein.

### GOVERNMENT FUNDING

The invention was made with government support under Contract No. ______ awarded by the United States Army. The government has certain rights in the invention.

### BACKGROUND

There have been numerous attempts to develop devices and methods for detecting hazardous materials, including chemical warfare agents (CWAs). A review of prior attempts can be found in the following article, the contents of which are incorporated herein in their entirety: “A Review of Chemical Warfare Agent (CWA) Detector Technologies and Commercial Off-The-Shelf Items”, Australian Government, Department of Defence, Defence Science and Technology Organisation (2009). Descriptions of other prior art methods of sensing hazardous materials can be found in U.S. Pat. Nos. 6,435,007 and 6,783,989, the contents of which are hereby incorporated by reference in their entirety. U.S. Pat. No. 6,435,007 describes a sensor system for monitoring breakthrough of a chemical agent through a vapor barrier using a carrier gas stream. U.S. Pat. No. 6,783,989 describes polymers, including conductive polymers, for use sensors for the detecting extremely hazardous substances, such as chemical warfare agents. U.S. Pat. No. 9,086,351 describes a device and method for detecting and quantifying permeation of a chemical through a glove. The contents of that patent are also hereby incorporated by reference in their entirety. Other descriptions of prior art devices and methods can be found in the following references: “Development of a Contact Permeation Fixture and Method” ECBC-TR-1141, Edgewood Chemical Biological Center, U.S. Army Research, Development and Engineering Command; and U.S. Pat. No. U.S. 9,021,865, each incorporated herein by reference in their entirety. These references describe one current method of testing nerve agents and other highly toxic chemicals using an Aerosol Vapor Liquid Assessment Group (AVLAG) cell.

Despite these prior efforts, there is a need for a device and method for sensitive, chemically-specific, real-time sensing of hazardous material whether that material is in the vapor or liquid phase. Further there is a need for a device and method of sensing penetration of hazardous materials through a barrier.

### SUMMARY OF THE INVENTION

In accordance with the foregoing objectives and others, one embodiment of the present invention provides a device for detecting permeation of a hazardous material through a test material. The device comprises a test cell having a first chamber configured to receive the hazardous material. The device also comprises a removable sensor module configured to hold the test material therein, and also configured to hold a removable sensor module. The removable sensor module comprises a sensor for detecting permeation of the hazardous material from the first chamber, wherein the sensor is comprised of a conductive polymer, a semi-conductive polymer or an electroactive polymer, the sensor being chemically reactive with the hazardous material to generate a change in electrical resistance in the sensor. The device further comprises one or more conductive electrodes attached to the sensor configured to detect a change in resistance in the sensor. In addition, the device comprises a resistance measuring device electronically connected to the one or more electrodes, the resistance measuring device configured to receive data from the one or more electrodes and generate an output based on the data corresponding to an amount of hazardous material detected by the sensing film.

Another embodiment of the present invention provides a method for detecting a hazardous analyte permeating through a test material in a test cell device having a first chamber and a second chamber. The method comprises receiving a removable sensor module between the first chamber and the second chamber, the removable sensor module comprising a sensing film comprising a conductive polymer, a semi-conductive polymer or an electroactive polymer that is chemically reactive with the hazardous analyte to generate a change in electrical resistance in the sensing film. The method also comprises continuously collect, using one or more conductive electrodes attached to the sensing film, data corresponding to changes in electrical resistance in the sensing film. The method further comprises analyzing the electrical resistance data of the sensing film to generate output corresponding to real-time concentrations of the hazardous analyte permeated from the first chamber to the second chamber.

In a further embodiment of the present invention, a sensor for detecting presence of a hazardous material in an environment is provided. The sensor comprises a sensing film for detecting presence of the hazardous material in the environment, wherein the sensing film is comprises a conductive polymer, a semi-conductive polymer or an electroactive polymer, the sensing film being chemically reactive with the hazardous material to generate a change in electrical resistance in the sensing film. The sensor also comprises a substrate comprising a non-conductive polymer, the substrate being configured to be in contact with or proximate to the sensing film such that a surface of the sensing film is exposed to the environment. The sensor further comprises one or more conductive electrodes attached to the sensing film configured to detect a change in resistance in the sensing film. Additionally, the sensor comprises a resistance measuring device electronically connected to the one or more electrodes, the resistance measuring device configured to receive data from the one or more electrodes and generate an output based on the data corresponding to an amount of hazardous material detected by the sensing film in real-time.

In a further embodiment, a method for real-time detection of a hazardous analyte in a remote location. The method comprises directing a remote-controlled device to enter the remote location, the device comprising a sensor comprising a sensing film for detecting presence of the hazardous analyte, wherein the sensing film comprises a conductive polymer, a semi-conductive polymer or an electroactive polymer, the sensing film being chemically reactive with the hazardous analyte to generate a change in electrical resistance in the sensing film. The method also includes continuously collect, using one or more conductive electrodes attached to the sensing film, data corresponding to changes in electrical resistance in the sensing film. The method further includes analyzing the electrical resistance data of the sensing film to generate output corresponding to real-time concentrations of the hazardous analyte at the remote location.

These and other aspects of the invention will become apparent to those skilled in the art after a reading of the following detailed description of the invention, including the figures and appended claims.

### BRIEF DESCRIPTION OF THE FIGURES

FIG. 1A illustrates a schematic diagram of a modified AVLAG test cell of the present invention in a sealed configuration;

FIGS. 1B1, 1B2, and 1B3 illustrate schematic diagram of a component of the modified AVLAG test cell of the present invention in a unassembled configuration

FIGS. 1C1, 1C2, and 1C3 illustrate schematic diagram of a component of the modified AVLAG test cell of the present invention in a unassembled configuration

FIGS. 1D1 and 1D2 illustrate schematic diagram of a cross-sectional view of component of the modified AVLAG test cell of the present invention.

FIG. 2. shows experimental data for Example I demonstrating breakthrough of dibenylamine through a 10 mm latext swatch.

FIGS. 3A and 3B show experimental data for the water proof sensors of Example II demonstrating sensitivity of sensor after total water immersion.

FIG. 4A and 4B show experimental data for Example III demonstrating breakthrough of 2-diethylaminoethanethiol through skin.

FIG. 5 shows experimental data for Example IV demonstrating breakthrough of nicotine through nitrile.

FIG. 6 shows experimental data for Example V demonstrating breakthrough of nicotine through cloth.

### DETAILED DESCRIPTION

This invention relates to a sensor for detecting and measuring the presence of a hazardous substance in real-time. The invention further relates to a sensor for detecting and measuring the penetration of hazardous substances through a barrier in real-time. Still further, this invention relates to a device, system and method for detecting and measuring the presence of a hazardous substance, and more particularly to a device, system and method for detecting and measuring the present of a chemical warfare agent (CWA). The devices, sensors and methods described herein provides real-time detection of amounts and/or concentrations of a hazardous analyte permeating through a test material (e.g., from a first chamber to a second chamber in a test cell device), which may be useful in providing improved monitoring of breakthrough of hazardous substances across protective materials, or in providing improved monitoring of unknown evironments, such as a combat zone. This monitoring may be performed for predetermined times or continuously over a period of time. The changes in concentration of hazardous analyte may be outputted in real-time, which can be used to provide life-saving alerts and/or prompt further action upon detection of hazardous materials, such as CWAs, simulants of CWAs and/or strong reducing agents above predetermined thresholds. Furthermore, the devices, sensors and methods described herein provides may be utilized to provide a continuous monitoring and detection of amounts and/or concentrations of a hazardous analyte. The sensors may detect changes in electrical resistence in the sensor continuously and provide a continuous output indictating real-time amounts and/or concentrations of the hazardous analyte detected over a period of time.

Sensor

One aspect of the present invention is a sensor for detecting the presence of a hazardous substance in real time. The sensor may be in contact or proximate to the substrate. In some embodiments, the sensor preferably has a substrate capable of containing, holding or supporting a sensing material which is capable of detecting the hazardous material. The detection event can then be read or converted into a form of information, like a signal, that can be read or transmitted in real time.

The substrate can be made of a material of any kind, but is preferably a polymer. Further, the substrate may be conductive or made from a non-conductive material such as glass, a ceramic, or a non-conductive polymer. The substrate can be rigid or flexible, and any size, shape or thickness. For some applications, the material is preferably a thin, flexible polymer. Optionally, the substrate may be surface modified by any suitable means, such as for example, a thin layer of imprinted metal, metal ions and/or complexes, e.g., zinc, gold copper, silver.

The sensing material can be any material capable of detecting the presence of hazardous materials. Preferably, the sensing material is chosen for its ability to detect the hazardous material or materials of interest and also the method of reading or transmitting the detection event. Factors such as the solubility and volatility of the hazardous material should be taken into account when choosing the sensing material. Other factors to consider include environment in which the sensing is to occur (e.g., temperature, humidity, etc.), and the phase of the hazardous material (e.g., solid, liquid, vapor, gas, aerosol). The sensing material can be formed into a film, array, or pattern.

The sensor may include any suitable materials capable of chemically reacting with the hazardous substance to be detected, and providing a detectable change, such as, for example, a change in electrical resistance in the sensing material. The sensor may be in contact with or proximate to (e.g., separated by a protective material) the hazardous substance to be measured. In one exemplary embodiment, the sensor may include a thin-film, such as a polymeric film, capable of chemically reacting with the targetted hazardous substance to generate a detectable change in the polymeric film. The thin-film may have any suitable thickness that is capable of reacting with the desired hazardous analyte. In particular, the thin-film may have a thickness from about 50 nm to about 200 nm, preferably about 100 nm. In a preferred embodiment, the detectable change may be in the form of a change in electrical resistance in the polymer. In some exemplary embodiments, the sensor may comprise, for example, a conductive polymer, a semi-conductive polymer, an electroactive polymer, and/or a non-conductive polymer. For example, conductive polymers are polymers whose backbones or pendant groups are responsible for the generation and propagation of charge carriers. These polymers typically exhibit dramatic changes in resistivity on exposure to certain chemical species. Many species have no effect on polymer resistivity. Typically, the resistivity of the virgin or doped conductive polymers decreases dramatically and irreversibly with exposure to dopant species. As another example, electroactive polymers are polymeric materials that conduct electricity. Chemical vapors interact with the polymer backbone, or a chemically reactive additive incorporated into the polymer, to produce a change (increase or decrease) in the electrical resistance of the polymer, which enables the polymer to function as a chemical sensor. A measurement in the change in polymer resistance provides an accurate quantification of the dose or concentration of a particular CWA, simulant of CWA, or strong reducing agent. For example, U.S. Pat. Nos. 5,310,507, 5,145,645, and 6,783,989, refer to several exemplary sensing materials such as conductive polymers. Suitable polymer for use as a sensor for detecting strong reducing agents and/or CWAs or simulants thereof include, but is not limited to, polyanilines, polyacetylenes, polydiacetylene, polypyrrole, polythiophene, and derivatives thereof. The strong reducing agents, CWAs and simulants of CWAs that may be detected by the sensor described herein include, for example, amines, sulfur and its derivatives, diols, and other strongly basic agents. In particular, the detector may include regioregular poly(3-hexylthiophene (irp3HT). The rrp3HT may be in the form of a coating or a film onto a substrate, and may be suitable for reacting with and detecting a number of different types of strong reducing agents, CWAs or simulants of CWAs, e.g., dibenzylamine, nicotine, 2-diethylaminoethanethiol, methyl salicylate, sulfur mustard, etc.

Researchers at the Massachusetts Institute have developed methods of sensing the presence of chemical warfare agents using chemiresistive sensors using carbon nanotubes. (“Carbon Nanotube/Polythiophene Chemiresistive Sensors for Chemical Warfare Agents,” J. AM. CHEM. SOC. 9 VOL. 130, NO. 16, 2008. The contents of these references are hereby incorporated herein by reference in their entirety.

The following reference details prior art relating to sensing, detection, decontamination and reactions of CWA's: Destruction and Detection of Chemical Warfare Agents, Chem. Rev., 2011, 111 (9), pp 5345-5403; Decontamination of Chemical Warfare Agents, Chem. Rev. 1992, 1729-1743. The contents of this article are hereby incorporated by reference in their entirety.

In a preferred embodiment of the present invention, the substrate is made from a non-conductive polymer and the sensing material is a conductive polymer, such as one of the conductive polymers listed in U.S. Pat. Nos. 5,310,507, 5,145,645, or 6,783,989; or from a semiconductive, or eletroactive polymer. The choice of conductive polymer is chosen to optimize detection of the specific analyte(s) of interest. The substrate with conductive polymer is coupled to one or more conductive electrodes which are then electrically connected to a resistance measuring device. The connection can be made through a wired connection, mobile or wireless connection, or any other means of communication or transmission.

The design and composition of this sensor may be modified to adjust for sensitivity, responsiveness, or environmental or other conditions. In one further preferred embodiment, the conductive electrodes are coated so as to have a tuned reduction oxidation potential. This coating would provide an advantage over prior art methods by reducing the need for incorporating additives or dopants to increase specificity.

In another embodiment, the sensor surface may be doped with a material selected to modify the electrical resistance of the sensing film. The dopant may be suitable for providing a redox reaction with the desired hazardous analyte, such as, for example, NOPF₆. Furthermore, the dopant may change the electrical resistance of the sensing film to any suitable range, such as for example, from about from about 500 to about 1000 ohm.

In further preferred embodiments of the present invention, the sensor is enhanced to perform better in humid environments, through the use of one or more of the following methods:
Use of hydrophobic conductive polymers to reduce degradation in humid environments (e.g., use longer side-chain polythiophenes—octyl as opposed to hexyl).
Coating the polymer sensor material surface with hydrophobic adlayers to reduce degradation in humid environments (e.g., coating by vapor deposition of fluorosilanes, silazanes, and silanes as well as spinning of ultrathin waxes or oils on the surface).
Mixing a hydrophobic component into polymer sensor material to reduce degradation in humid environments (e.g., wax of fluoropolymer added in the solution used to spin the polymer)
Use of microporous membranes, for example those mentioned in U.S. Pat. No. 6,783,989.
Pre-treating surface with a hydrophobic, hydrophilic, acidic, basic or reactive coating
Coating sensor with absorbent layer, e.g., MOF or carbon

For example, the sensor may be further coated with a silcone polymer, such as for example, a polysiloxane, to provide a separation of the sensor material from the environment, in particular a humid or moist environment. The silicone coating may impart improved water resistance or water proofness to the sensor.

Other potential modifications to the sensor of the present invention include:
Modifying the surface structure/morphology
   Modifying the surface texture to optimize wetting property of the surface
      Increase surface area
      Enhance hydrophilicity or hydrophobicity
   Protecting sensor with a semi-permeable membrane or polymer layer
   Coating with a thin layer of molecular imprinted polymer to achieve selectivity
Modifying the sensor surface
   Modifying the surface of the conductive polymer
      Plasma treatment, ozone treatment
      Silane treatment (immediately after plasma treatment)
   Modifying the surface to have the following attributes:
      Hydrophobicity, e.g. hydrophobic silane treatment
      Hydrophilicity, e.g. PEG silane treatment
      Reactivivity: e.g. carboxylate, amine, oxime, zinc, epoxide
      Bioactivity: e.g. enzyme, antibody
   Coating the surface with a thin layer, e.g. crosslinked PEI, oxime-functionalized PEG, siloxane, silwet, PVA, reactive nanoparticles
   Coating the surface with a porous layer: e.g. silica, metal oxide, MOF, carbon, porous polymer layer, cyclodextrin
Incorporating additives
   Oxime derivatives: pyridine aldoxime, pralidoxime, 4-dimethylaminopyridine
   Metal, metal ions, complexes, e.g. zinc, gold, copper, silver.
   Plasticizer
   Amphiphlic polymers, e.g. Irgsurf, alkylamine, alkyl oxime, block copolymer (polystyrene-co-PEG, polymethacrylate-co-PEG)
   Polymeric acid, PEG, polyamine, polystyrene sulfonic acid, polyacrylic acid, polyquaternary ammonium materials
Incorporating bulk modification (polymer modification)
   Various functional groups can be attached to the conductive polymer backbone, e.g. the functional group on the 3-position of the polythiophene including PEG, carboxylic acid, sulfonic acid, amine, hydroxyl, oxime, imidazolium, and siloxane
   Various doping acid for polyaniline based sensor
Using surface patterning to create a multifunctional sensor (Dosimetric electronic noses)
   Can develop an array of sensors by patterning the surface of the conductive polymer materials
      e.g. hydrophobic vs. hydrophilic
      Basic vs. acidic
      Amine, acid, oxime, enzyme
   Each section can have different detection capabilities due to differences in wetting, adsorption, chemical interaction and reactions
Modifying sensors for particulate contaminants including solid dusty agents and aerosols
   Modify sensor to have highly porous layer
   Modify sensor to have a charged surface layer
   Modify sensor to have a hydrophobic gel layer
   Modify sensor to have a hydrophilic gel layer
   These surface layers can attract and dissolve solid and aerosol particles

Sampling

Due to the danger of exposure to hazardous materials, there is also a need in the art for a sensor that can be delivered and retrieved remotely, without the need to directly expose a human to the site at which the sensing or detection is to occur. Another aspect of the present invention is sensing or detection through the use of a sensor delivery device such as robot, drone, remote controlled mobile vehicle, Unmanned Ground Vehicle (UGV), Unmanned Aerial Vehicle (UAV) or any other means of delivering the sensor to and retrieving the sensor from the sensing/detection site. The sensor described above can be attached to, mounted on, or incorporated within the sensor delivery device. Such device or vehicle can be delivered to and retrieved from the site of sensing or detection using human control or through programmed control, machine learning-derived control, artificial intelligence-derived control or otherwise.

The sensor delivery device such as robot, drone, remote controlled mobile vehicle, Unmanned Ground Vehicle (UGV), Unmanned Aerial Vehicle (UAV) or any other means of delivering the sensor in combination with one or more of the senors can provide unmanned, remote controled, real-time analysis of a sensing/detection site, which the sensor delivery device is still located at the site. The sensor may chemically react with a hazardous analyte at the sensing/detection site, and the resistance measuring device may detect a change in electrical resistance in the sensor and wirelessly transmit data corresponding to the change in electrical resistance to a remotely located computational device. The computational device may be located with a user within a known safe region, while the sensor delivery device is remotely controlled by the user to explore unknown sites.

The sensor delivery device can be a part of or used in connection with UAV's serving other purposes such as the following:
Target and decoy—simulating an enemy aircraft or missile
Reconnaissance—providing battlefield intelligence
Combat—providing attack capability
Research and development—developing technologies
Civil and Commercial UAV's

There is also a need in the art for a sensing device that is also capable of retrieving a sample of the potentially hazardous material from one site and delivering the sample to a different site for further testing. Sampling means, including but not limited to robotic hands, scoopers, swabbers, and adhesive contact pads can be used to grab or otherwise collect a sample. The sampling means can be attached or connected to, mounted on, or incorporated within the sensor or sensor delivery devices described above.

The following reference describes method and device for remote sampling of hazardous materials: “Remote chemical biological and explosive agent detection using a robot-based Raman detector”, Proc. SPIE 6962, Unmanned Systems Technology X, 69620T (Apr. 16, 2008); doi:10.1117/12.781692. The contents of that reference are incorporated by reference herein in their entirety.

The following reference describes an aerosol sample detection system that is coupled to an aerial vehicle with sample collection capability: U.S. Pat. No. 6,854,344. The contents of that reference are incorporated by reference herein in their entirety.

Measuring breakthrough

Due to the danger of exposure to hazardous materials, including CWA's, there is a need for a better sensor system for testing breakthrough or penetration of a CWA through a barrier. The risk of penetration through chemical suits, masks and filters intended to shield people and equipment is sever. One aspect of the present invention is a sensor and sensor system for detecting and measuring such breakthrough.

The following references describe prior art methods of testing for breakthrough of a hazardous material through a barrier:
“Standard Guide for Documenting the Results of Chemical Permeation Testing of Materials Used in Protective Clothing”, American Society for Testing and materials, West Conshohocken, Pa., 19428, reprinted from the Annual Book of ASTM Standards, Copyright ASTM
“Standard Test Method for Resistance of Protective Clothing Materials to Permeation by Liquids or Gases Under Conditions of Continuous Contact”, American Society for Testing and materials, West Conshohocken, Pa., 19428, reprinted from the Annual Book of ASTM Standards, Copyright ASTM.
“Standard Guide for Selection of Chemicals to Evaluate Protective Clothing Materials” American Society for Testing and materials, West Conshohocken, Pa., 19428, reprinted from the Annual Book of ASTM Standards, Copyright ASTM.
“Standard Classification System for Chemicals According to Functional Groups”, American Society for Testing and materials, West Conshohocken, Pa., 19428, reprinted from the Annual Book of ASTM Standards, Copyright ASTM.
“Standard Test Method for Resistance of Materials Used in Protective Clothing to Penetration by Liquids”, American Society for Testing and materials, West Conshohocken, Pa., 19428, reprinted from the Annual Book of ASTM Standards, Copyright ASTM.
U.S. Pat. No. 6,435,007

The contents of those references are incorporated by reference herein in their entirety.

Some of these prior art methods rely on the collection, subsequent analysis and calculation of breakthrough and breakthrough time. Others rely on a carrier gas to facilitate penetration through a barrier. The sensor system of the present invention provides real-time detection and measurement of breakthrough, without the need to employ a carrier gas.

One device of the present invention for measuring breakthrough is a multi-chambered cell designed to hold a piece of material as an interface between at least two chambers; wherein a chemical is placed on the material in one chamber and a sensor capable of sensing the chemical is placed on the opposite side of the material in a second chamber and can detect when a chemical has traversed through the material from one side to the other. The sensor can be of the type described above. A preferred sensor is made of a non-conductive polymer coated with a conductive polymer. In a further preferred embodiment, the substrate is made of mylar and the sensor material is a conductive polymer. This flexible configuration can be used to measure penetration through flexible barrier materials, such as fabric, and can be incorporated between layers of barrier materials.

For example, the device may be configured to measure breakthrough of one or more layers of test materials, said test material may comprise barrier materials and/or protective materials against hazardous agents (e.g., CWAs, simulants of CWAs, and other strong reducing agents as discussed above). In another example, the device may include a plurality of sensors and/or senor modules interspersed between multiple layers of test materials, e.g., barrier materials and/or protective materials. In particular, the sensors and layers of test materials may be interdigitating articles having a plurality of layer. At least one sensor may be placed to one side of the interdigitating article. In another embodiment, a plurality of sensors may be interspersed between multiple layers of test materials such that breakthrough may be measured for each intermediary and/or additional layer. Furthermore, the one or more layers of test materials may be in any suitable configuration and/or geometry in two-dimensional or three-dimensional space. For example, the layers of test materials may be in the form of stacked layers of sheets. In another example, the layers of test materials may be in the form of nested three-dimensional shapes, e.g., nesting spheres, cylinders, or other three dimensional regular or irregular shapes.

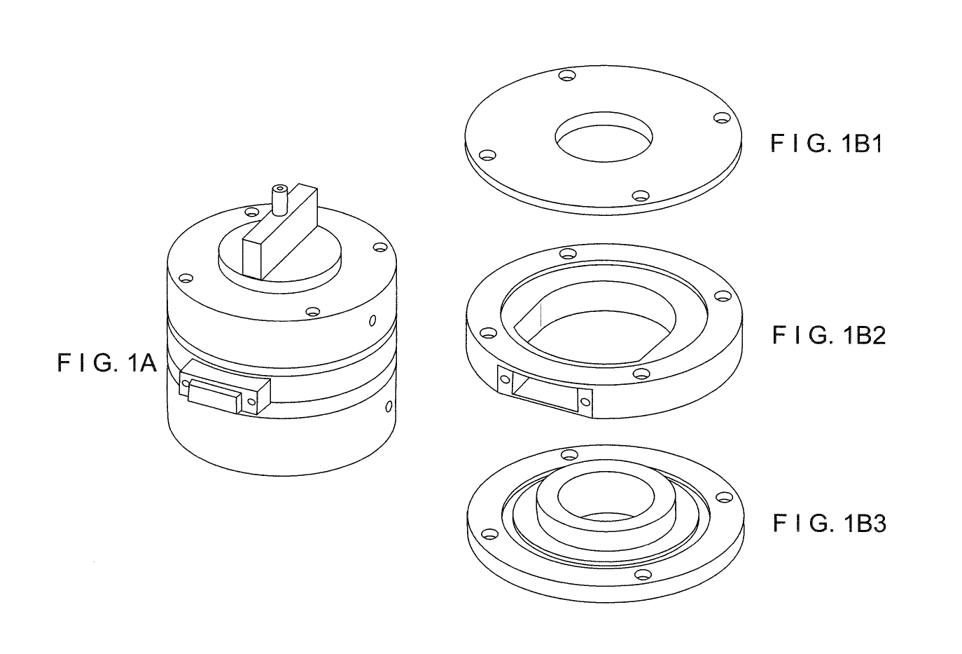

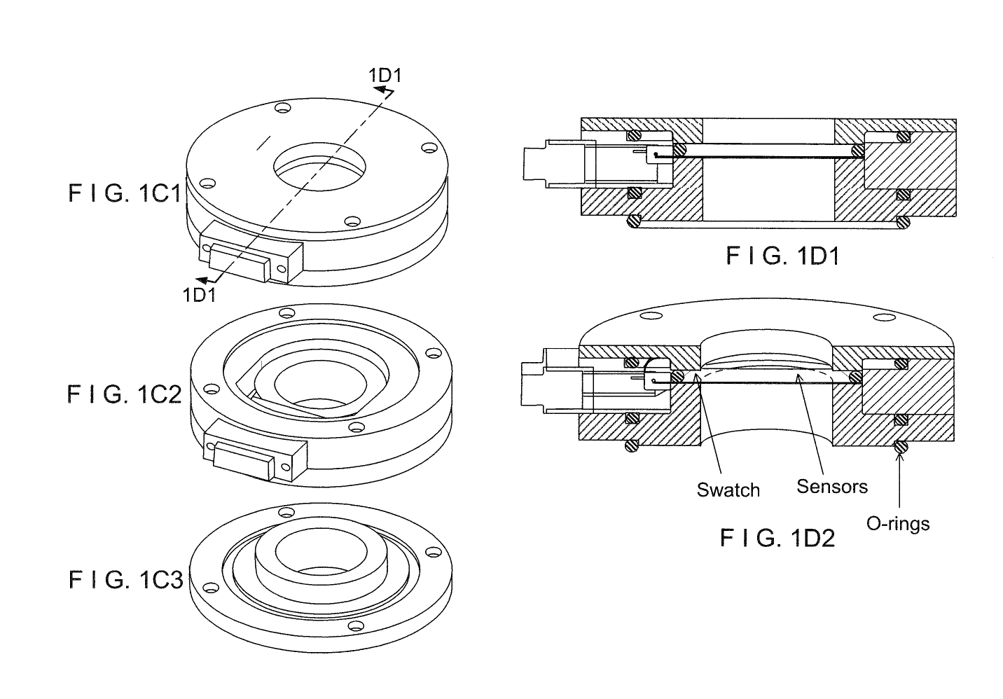

One aspect of the present invention is an improved testing device, as shown in FIGS. 1A through 1D. In this new device, the AVLAG cell has top plate, lower plate, connector and connector plate. The connector is capable of receiving and holding one or more sensors. In a preferred embodiment, the testing device of the present invention is constructed such that the connector and sensors are removable. (For example, by a removable sensor chip that can slide in and out of a slot in the AVLAG cell). Furthermore, test cell of the present invention does not require the use of vacuum, application of weights to compress the testing material and the sensor together, or other modifications to apply a pressure between the sensor and the test material. Rather, the sensor may be placed in contact with or proximate to the test materials. Furthermore, contrary to conventional AVLAG cells, changes to the sensor, particularly changes to the electrical resistance in the sensor, may be measured while the removable sensor module remains in the test cell and provide analytical data in real-time, and does not require a separate step of removing the sensor module from the test cell and transporting it to a separate analytical device for a subsequent and delayed analysis. In addition, such a removal and transport of the sensor, further exposes the sensor to environmental factors, e.g., humidity or contaminants, that may reduce accurracy or reliability of the sensor. Thus, the present invention provides a single device (e.g., unitary device) that is configured to expose the sensor to a hazardous analyte, and detect the amount of hazardous analyte that permeates through the test material or layers of test materials.

In particular, the improved testing device may include a removable sensor module that modularly provides a sensor which reacts specifically to a desired analyte. The removable sensor may be easily removed and replaced with a different sensor module to allow for detection of different types of CWAs depending on they sensor module used. The sensor module may include a sensor as described above having a polymer that is chemically reactive with the desired hazardous analyte to generate a change in electrical resistance in the sensor. As can be seen in FIGS. 1A through 1D2, the removable sensor module may be configured to hold a swatch of the test material therein, as well as a sensing material for detection of the desired hazardous analyte. The amount of hazardous analyte permeating through the test materal may be measured using the test device in real-time, without delay from removal and separate testing of the sensor, after it has been exposed to the hazardous analyste. Instead, the removable sensor module may remain in the testing device while simultaneously providing data to a resistance measuring device to generate an output based on the data corresponding to real-time changes in amount or concentration of the hazardous analyte detected by the

[symbol]

During use, a swatch of material is positioned on top of the sensor, between the sensor and the O-ring. The hazardous agent to be tested is then placed in the testing cell in a manner similar to that employed in using current AVLAG testing cells.

The sensor of the present invention is a (device) having a surface made with or having a surface coated, in whole or in part, with an indicator material which indicates a conductivity change in the presence of certain hazardous chemical compounds. This indicator material may be any material capable of indicating a conductivity change visually, electrochemically, or otherwise, such materials including but not limited to those described in U.S. Pat. No. 6,783,989, the contents of which are hereby incorporated herein in their entirety. Preferably, the sensor is made of a polymer coated in part by a conductive polymer.

### EXAMPLES

### Example 1: Latex Breakthrough Testing

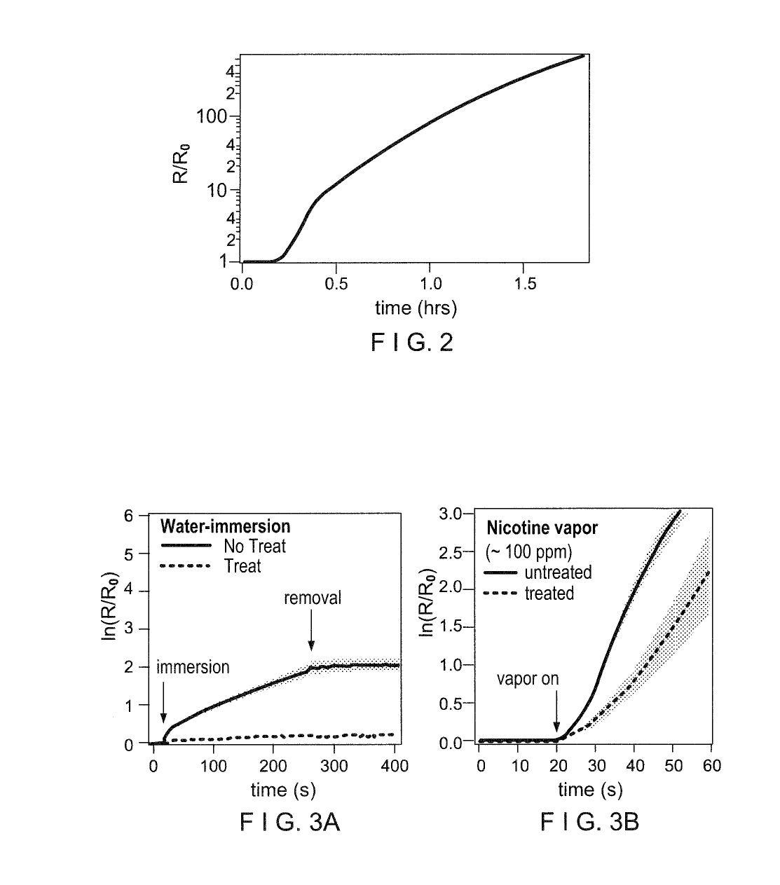

In one exemplary embodiment, a device for detection of breakthrough or permeation of a hazardous material may be provided. The test material in Example is a 10 mil latex swatch and breakthough of a hazardous analyte, i.e., dibenylamine over time is determined using a test cell device. FIG. 2 shows a plot of resistance (relative to the initial resistance, R/Ro) versus time for the breakthrough of dibenzylamine through a 10 mil latex swatch. The latex swatch was placed on top of the thin-film sensor and one microliter of dibenzylamine was added to the swatch.

### Example II: Waterproofed sensors

In another embodiment, a substantially waterproof sensor for detection of a hazardous material may be provided. A thin regioregular poly(3 hexyl thiophene) (rrP3HT) film over interdigitated electrodes was coated with fluorinated silane by vapor deposition for 1 hour. This film was doped with NOPF₆ in acetonitrile to a resistance of between 500 and 1000 ohms. This film showed enhanced resistance to dedoping when placed into water versus the film not containing the fluorinated silane coating (see FIG. 3A). Although the film confers resistance to moisture, it does not completely block the film and still response to nicotine vapor with only a 2-fold decrease in response rate (see FIG. 3B).

Details:

### Sensor:

Substrate: 4 mil Mylar with 70 nm-thick patterned gold
Electrode Geometry: 8.65 mm² area of 20 μm width and 20 μm spaced interdigitated electrodes
Coating: 100 nm thick rrP3HT film
Post-treatment: vapor treatment of (heptadecafluoro-1,1,2,2-tetrahydrodecyl)trichlorosilane for 1 hour at room temperature under vacuum
Doping: with NOPF₆ in acetonitrile to a resistance between 500 and 1000 ohm

### Sensing Geometry

Resistance measurements plotted as natural logarithm of resistance divided by the initial resistance
Sensor hooked into flat flexible connector attached to Keithley 2700 with multiplexing unit
Sensor dipped into beaker containing deionized water and removed after a few minutes (see FIG. 3A)
Sensor placed in enclosure with a nicotine vapor of approximately 100 ppm concentration (see FIG. 3B)

Experimental Results

FIG. 3A. Sensor response to total water immersion without (black) and with (red) perfluorosilane treatment. FIG. 3B. Average sensor responses to ˜100 ppm of nicotine vapor without (black) and with (red) treatment with the silane. There is a moderate decrease of roughly a factor of two in sensitivity after treatment. Curves are averages of several sensors run at the same time (shaded areas indicate one standard deviation from the mean).

### Example III: Breakthrough of an Amine with Skin

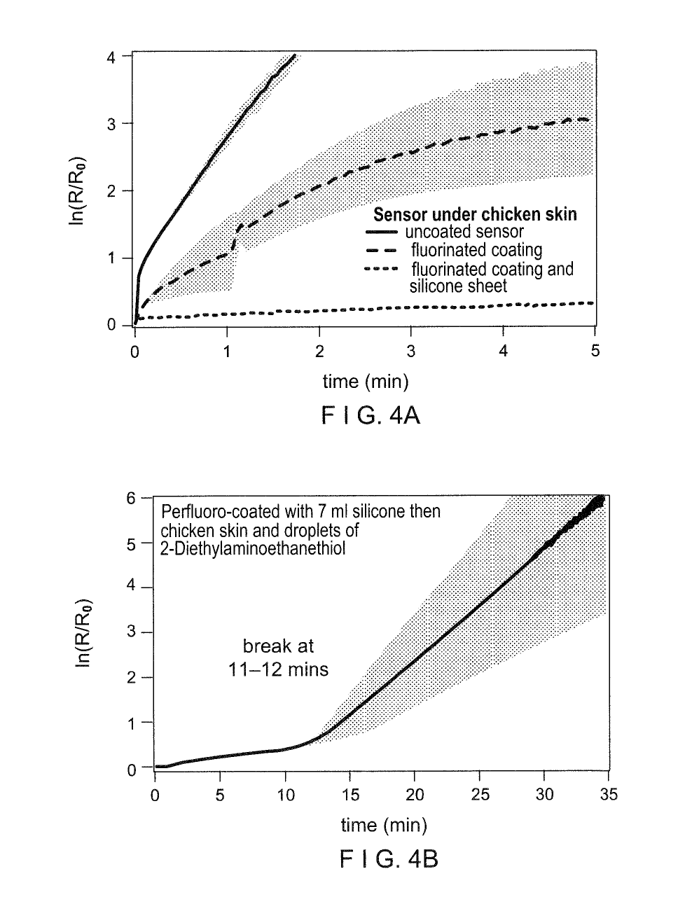

The presence of water and other chemicals can dedope conductive polymers over time. The addition of a fluorinated silane coating helps reduce the effect that moisture has on the sensor (see FIGS. 3A and 3B), but presence of other contaminants in biological samples can still alter the sensor response. We took the waterproofed sensor formulation from FIG. 3A and covered it with a 7-mil thick sheet of silicone. We tested this sensor and variants without the coatings by placing them under chicken skin (from raw chicken breast, see FIGS. 4A and 4B). The resulting sensor covered with silicone is only marginally reactive toward the chicken skin. A separate experiment (not shown) indicates that droplets of the simulant (2-diethylaminoethanethiol) are retarded by less than 20 seconds from reaching the sensor with the silicone coating. We tested the breakthrough of 2-diethylaminoethanethiol through chicken skin using the silicone-coated sensor. The breakthrough measurement shows an obvious upturn at roughly 11 to 12 minutes after adding the droplets.

Details:

### Sensor:

Substrate: 4 mil Mylar with 70 nm-thick patterned gold
Electrode Geometry: 8.65 mm² area of 20 μm width and 20 μm spaced interdigitated electrodes
Coating: 100 nm thick pure rrP3HT film
Post-treatment: vapor treatment of (heptadecafluoro-1,1,2, 2-tetrahydrodecyl)trichlorosilane for 1 hour at room temperature under vacuum
Doping: with NOPF₆ in acetonitrile to a resistance between 500 and 1000 ohm
Subsequent layer: 7-mil silicone sheet

### Sensing Geometry

Resistance measurements plotted as natural logarithm of resistance divided by the initial resistance
Sensor hooked into flat flexible connector attached to Keithley 2700 with multiplexing unit
Sensor placed under chicken skin (from raw chicken breast)
For break measurements, 1 uL droplets of 2-diethylaminoethanethiol are placed over every sensor surface

Experimental Results

FIG. 3A. Effect of various coatings on rrP3HT on the baseline response when placed under chicken skin. FIG. 3B The sensor with the fluorinated coating and silicone sheet was used in a breakthrough experiment using droplets of 2-diethylaminoethanethiol on chicken skin. An upturn in the response at 11-12 minutes indicates break. Curves are averages of several sensors run at the same time (shaded areas indicate one standard deviation from the mean).

### Example IV: Breakthrough of Nicotine with Nitrile

Breakthrough curves for a variety of simulants can be obtained. Here we show a break curve for nicotine through glove material—the palm area from a 4-mil nitrile glove.

Details:

### Sensor:

Substrate: 4 mil Mylar with 70 nm-thick patterned gold
Electrode Geometry: 8.65 mm² area of 20 μm width and 20 μm spaced interdigitated electrodes
Coating: 100 nm thick pure rrP3HT film
Post-treatment: none
Doping: with NOPF6 in acetonitrile to a resistance between 500 and 1000 ohm

### Sensing Geometry

Resistance measurements plotted as natural logarithm of resistance divided by the initial resistance
Sensor hooked into flat flexible connector attached to Keithley 2700 with multiplexing unit
Sensor placed under 4-mil thick nitrile from the palm area
1 uL droplets of nicotine are placed over every sensor surface

Experimental Results

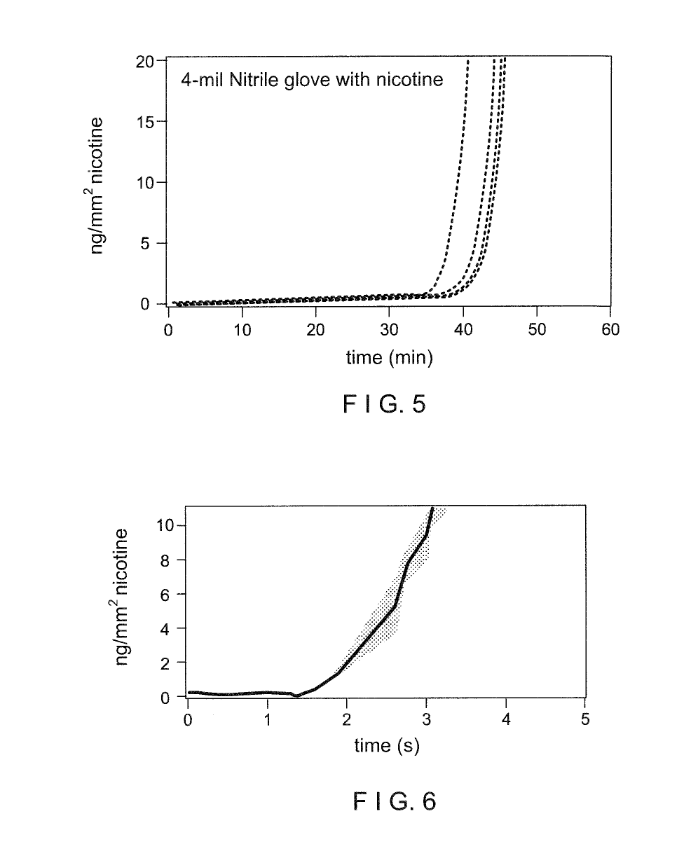

FIG. 5. Break curve (calibrated) of nicotine through 4-mil glove Nitrile. Challenge is 1 microliter drop over each sensor surface. Each curve is from a single sensor face (four in total).

### Example V: Breakthrough of Nicotine through Cloth

Breakthrough curves for a variety of simulants can be obtained. Here we show a break curve for nicotine through fabric—a 50/50 nylon cotton blend of fabric. The nicotine in this case is applied by a nicotine patch (NicoDerm CQ).

Details:

### Sensor:

Substrate: 4 mil Mylar with 70 nm-thick patterned gold
Electrode Geometry: 8.65 mm² area of 20 μm width and 20 μm spaced interdigitated electrodes
Coating: 100 nm thick pure rrP3HT film
Post-treatment: none
Doping: with NOPF₆ in acetonitrile to a resistance between 500 and 1000 ohm

### Sensing Geometry

Resistance measurements plotted as natural logarithm of resistance divided by the initial resistance
Sensor hooked into flat flexible connector attached to Keithley 2700 with multiplexing unit
Sensor placed under 50/50 nylon cotton fabric
Nicoderm CQ patch placed over fabric

Experimental Results

FIG. 6. Break curve (calibrated) of nicotine patch through 50/50 nylon cotton. Curve is average of six sensor faces. Shaded area indicates one standard deviation from the mean.

The invention described and claimed herein is not to be limited in scope by the specific embodiments herein disclosed since these embodiments are intended as illustrations of several aspects of this invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All publications cited herein are incorporated by reference in their entirety.

1. A device for detecting permeation of a hazardous material through a test material, comprising:
a test cell having a first chamber configured to receive the hazardous material;
a removable sensor module configured to hold the test material therein, and also configured to hold a removable sensor module comprising:
a sensor for detecting permeation of the hazardous material from the first chamber, wherein the sensor is comprised of a conductive polymer, a semi-conductive polymer or an electroactive polymer, the sensor being chemically reactive with the hazardous material to generate a change in electrical resistance in the sensor;
one or more conductive electrodes attached to the sensing film configured to detect a change in resistance in the sensing film; and
a resistance measuring device electronically connected to the one or more electrodes, the resistance measuring device configured to receive data from the one or more electrodes and generate an output based on the data corresponding to an amount of hazardous material detected by the sensing film.

2. The device of claim 1, wherein the sensor is comprised of a polymeric film.

3. The device of claim 1, wherein the sensor comprises a polymer that is irreversibly reactive with the hazardous material.

4. The device of claim 1, wherein the sensor comprises a hydrophobic conductive polymer.

5. The device of claim 4, wherein the hydrophobic conductive polymer is selected from a group consisting of polyaniline, polyacetylene, polydiacetylene, polypyrrole, polythiophene, and derivatives thereof.

6. The device of claim 5, wherein the sensor comprises a film comprising regioregular poly(3-hexyl-thiophene).

7. The device of claim 1, wherein the sensor further comprises a hydrophobic adlayer comprising a material selected to reduce degradation of the sensor in humid environments.

8. The device of claim 7, wherein the hydrophic adlayer comprises a vapor deposition coating onto the sensor comprising one or more of fluorosilanes, silazanes, and silanes.

9. The device of claim 8, wherein the hydrophic adlayer comprises (heptadecafluoro-1,1,2,2-tetrahydrodecyl) tricholorsilane.

10. The device of claim 1, wherein the sensor is doped with a material selected to modify the electrical resistance of the sensor.

11. The device of claim 10, wherein the sensor is doped with NOPF₆ to modify the electrical resistance of the sensor to from about 500 to about 1000 ohm.

12. The device of claim 1, wherein the sensor comprises a mixture of a hydrophobic component, and one of the conductive polymer, the semi-conductive polymer or the electroactive polymer.

13. The device of claim 1, wherein the mixture further comprises one or more additives selected from the group consisting of: an oxime derivative, a metal, a metal ion, a metal complex, a plasticizer, an amphiphlic polymer, a polymeric acid, polyethylene glycol, a polyamine, a polysterene sulfonic acid, a polyacrylic acid, and a polyquanternary ammonium.

14. The device of claim 1, wherein the sensing film comprises a microporous membrane.

15. The device of claim 1, wherein the sensor further comprises a hydrophobic, hydrophilic, acidic, basic or reactive coating.

16. The device of claim 1, wherein the one or more conductive electrodes are configured to be in communication with a resistance measuring device via communications network.

17. The device of claim 16, the one or more conductive electrodes are configured to be in communication with the resistance measuring device via a wired connection, a mobile connection or a wireless connection.

18. A method for detecting a hazardous analyte permeating through a test material in a test cell device having a first chamber and a second chamber, comprising:
receiving a removable sensor module between the first chamber and the second chamber, the removable sensor module comprising a sensing film comprising a conductive polymer, a semi-conductive polymer or an electroactive polymer that is chemically reactive with the hazardous analyte to generate a change in electrical resistance in the sensing film;
continuously collect, using one or more conductive electrodes attached to the sensing film, data corresponding to changes in electrical resistance in the sensing film; and
analyzing the electrical resistance data of the sensing film to continuously generate output corresponding to real-time concentrations of the hazardous analyte permeated from the first chamber to the second chamber.

19. The method of claim 18, wherein the hazardous analyte is a chemical warfare agent (CWA), a simulant of a CWA, or a strong reducing agent.

20. The method of claim 19, wherein the hazardous analyte is selected from a group consisting of amines, sulfur and its derivatives, diols, and strongly basic agents.

21. The method of claim 19, wherein the hazardous analyte is selected from a group consisting of 2-diethylaminoethanethiol, methyl salicylate (MeS) and sulfur mustard (HD).

22. A sensor for detecting presence of a hazardous material in an environment, comprising:
a sensing film for detecting presence of the hazardous material in the environment, wherein the sensing film is comprising a conductive polymer, a semi-conductive polymer or an electroactive polymer, the sensing film being chemically reactive with the hazardous material to generate a change in electrical resistance in the sensing film;
a substrate comprising a non-conductive polymer, the substrate being configured to provide structural support to the sensing film such that a surface of the sensing film is exposed to the environment;
one or more conductive electrodes attached to the sensing film configured to detect a change in resistance in the sensing film; and
a resistance measuring device electronically connected to the one or more electrodes, the resistance measuring device configured to receive data from the one or more electrodes and generate an output based on the data corresponding to an amount of hazardous material detected by the sensing film in real-time.

23. The sensor of claim 22, wherein the sensing film is further coated with a silicone coating to impart moisture resistance to sensing film.

24. A method for real-time detection of a hazardous analyte in a remote location comprising:
directing a remote-controlled device to enter the remote location, the device comprising a sensor comprising a sensing film for detecting presence of the hazardous analyte, wherein the sensing film comprises a conductive polymer, a semi-conductive polymer or an electroactive polymer, the sensing film being chemically reactive with the hazardous analyte to generate a change in electrical resistance in the sensing film;
continuously collect, using one or more conductive electrodes attached to the sensing film, data corresponding to changes in electrical resistance in the sensing film; and
analyzing the electrical resistance data of the sensing film to continuously generate output corresponding to real-time concentrations of the hazardous analyte at the remote location.

25. The method of claim 24, wherein the hazardous analyte is a chemical warfare agent (CWA), a simulant of a CWA, or a strong reducing agent.

26. The method of claim 24, wherein the hazardous analyte is selected from a group consisting of amines, sulfur and its derivatives, diols, and strongly basic agents.

27. The method of claim 24, wherein the hazardous analyte is selected from a group consisting of 2-diethylaminoethanethiol, methyl salicylate (MeS) and sulfur mustard (HD).
